(19) United States
(12) Patent Application Publication
(10) Pub. No.: US 2026/0355322 A1
(43) Pub. Date: Nov. 12, 2026

(54) DEVELOPMENT OF A NOVEL EBNA-1 DEGRADER TARGETING THROUGH MDM2

(71) Applicant: THE WISTAR INSTITUTE OF ANATOMY AND BIOLOGY, Philadelphia, PA (US)

(72) Inventors: Paul LIEBERMAN, Philadelphia, PA (US); Joseph SALVINO, Philadelphia, PA (US)

(73) Assignee: THE WISTAR INSTITUTE OF ANATOMY AND BIOLOGY, Philadelphia, PA (US)

(21) Appl. No.: 19/106,277

(22) PCT Filed: Aug. 30, 2023
(86) PCT No.: PCT/US2023/073156

§ 371 Date: Feb. 25, 2025

Related U.S. Application Data
(60) Provisional application No. 63/402,443, filed on Aug. 30, 2022.

(51) Int. Cl. A61K 47/55; C07D 209/14; C07D 209/18

(52) U.S. Cl. CPC A61K 47/55; C07D 209/14; C07D 209/18

(57) ABSTRACT

Disclosed herein are compounds of the formulas: as well as analogs thereof, wherein the variables are defined herein. Also provided are pharmaceutical compositions thereof. In some aspects, the compounds and compositions provided herein may be used to inhibit EBNA-1. Also provided are methods of administering compounds and compositions provided herein to a patient in need thereof, for example, for the treatment or prevention of autoimmune and EBV-related cancers.

This application claims the benefit of priority to U.S. Provisional Application 63/402,443, filed Aug. 30, 2022, the entire contents of which are hereby incorporated by reference.

This invention was made with government support under CA259171, awarded by the National Institutes of Health. The government has certain rights in the invention.

### BACKGROUND

### I. Field

This disclosure relates to the fields of biology, pharmacology, medicine, and chemistry. In particular, new compounds, compositions, and methods of treatment related to the treatment of autoimmune diseases and viral associated cancers are disclosed.

### II. Description of Related Art

Inflammatory diseases, particularly autoimmune diseases, frequently have severe and long-term adverse effects on physical well-being and quality of life. In many patients these diseases cause significant disability, and in some cases, they may be life-threatening. Epstein-Barr virus (EBV or human herpesvirus 4, HHV-4) has been associated with both autoimmune diseases and viral associated cancers, such as Burkitt's lymphoma, non-Hodgkin's lymphoma and nasopharyngeal carcinoma (Andrei, et al.). Central nervous system (CNS)-related diseases such as MS and PCNSL have been linked to EBV as well (Gandhi, et al.). Recent advances in therapeutic options have improved outcomes and quality of life for many patients; however, significant numbers of patients do not achieve adequate relief of symptoms from these therapies or cannot tolerate them. Even in patients who do respond, side effects can be significant and may be life-threatening due to immune suppression or other complications. Therefore, small molecules that target EBV latent infection represent a significant and urgent unmet medical need for use in the treatment of EBV-associated cancers and autoimmune disorders.

Besides EBERs and mi-RNAs, EBNA-1 is the only protein expressed in all forms of latency during an EBV infection (Frappier et al.). It localizes to the nucleus and has multiple roles, including binding to cellular and viral genomes, regulation of signaling pathways, and gene transcription. EBNA-1 is also thought to act as an oncoprotein and links EBV infection to carcinogenesis (Boudreault, et al.). Specifically, EBNA-1 induces chromosomal abnormalities by activating the transcription of the catalytic subunit of the NADPH oxidase, NOX2/gp91^{Phox}, increasing the production of reactive oxygen species (ROS) (Gruhne et al.). EBNA-1-induced increase in ROS can be linked to the development of genomic instability and may thus contribute to the multistep process of tumor development (Schultz et al.). Because EBNA-1 is expressed in all EBV-associated tumors, it could represent a common mechanism in EBV-induced oncogenesis.

To this end, attempts have been made to inhibit EBNA-1 by targeting the protein for degradation. For example, the several small molecule targets that bind to and inhibit EBNA-1 through H-bond, as well as π-alkyl, π-σ, π-cation, unfavorable donor-donor, and halogen interactions have recently been identified (Jakhmola et al. and Messick et al.). Also, an x-ray crystal structure of the reported EBNA-1 inhibitor, VK-0941 in complex with EBNA-1 was recently solved by the Lieberman lab. However, there is a strong deficiency of pharmaceuticals with the ability to treat EBV-related diseases and cancers.

Therefore, there remains a need to develop further compounds that may be used to target EBNA1.

### SUMMARY

In some aspects, the present disclosure provides compounds, pharmaceutical compositions, and methods for their use in the treatment of autoimmune diseases and viral associated cancers. In particular, the compounds may be formulated as a PROTAC and target EBNA1. In some embodiments, the compounds are further defined as:

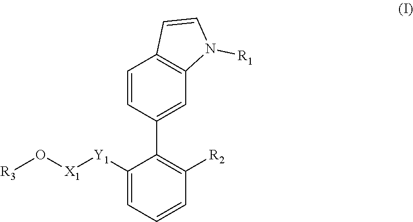

wherein:
R₁ is hydrogen, alkyl_{(C≤8)}, or substituted alkyl_{(C≤8)};
R₂ is —C(O)Rₐ, wherein:
   Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
   R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
   X₂ is O or NR_{d}, wherein R_{d} is hydrogen, alkyl_{(C≤8)}, or substituted alkyl_{(C≤8)};
Y₁ is alkanediyl_{(C≤12)}, cycloalkanediyl_{(C≤12)}, alkenediyl_{(C≤12)}, alkynediyl_{(C≤12)}, or a substituted version thereof;
X₁ is arenediyl_{(C≤12)}, heteroarenediyl_{(C≤12)}, or a substituted version thereof; and
R₃ is hydrogen, alkyl_{(C≤3)}, substituted alkyl_{(C≤8)}, or a group of the formula:

—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

In some embodiments, the compounds are further defined as:

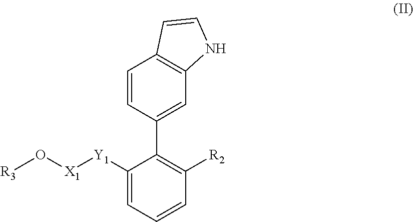

wherein:
R₂ is —C(O)Rₐ, wherein:
   Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
   R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
   X₂ is O or NR_{d}, wherein R_{d} is hydrogen, alkyl_{(C≤8)}, or substituted alkyl_{(C≤8)};
Y₁ is alkanediyl_{(C≤12)}, cycloalkanediyl_{(C≤12)}, alkenediyl_{(C≤12)}, alkynediyl_{(C≤12)}, or a substituted version thereof;
X₁ is arenediyl_{(C≤12)}, heteroarenediyl_{(C≤12)}, or a substituted version thereof; and
R₃ is hydrogen, alkyl_{(C≤8)}, substituted alkyl_{(C≤8)}, or a group of the formula:

—Y₂—C(O)Rₑ
wherein:
   Y₂ is alkanediyl_{(C≤12)} or substituted alkanediyl_{(C≤12)}; and
   Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
      or a pharmaceutically acceptable salt thereof.
      In some embodiments, the compounds are further defined as:

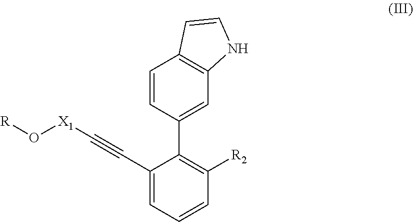

wherein:
R₂ is —C(O)Rₐ, wherein:
   Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
   R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
   X₂ is O or NR_{d}, wherein R_{d} is hydrogen, alkyl_{(C≤8)}, or substituted alkyl_{(C≤8)};
   X₁ is arenediyl_{(C≤12)}, heteroarenediyl_{(C≤12)}, or a substituted version thereof; and
R₃ is hydrogen, alkyl_{(C≤8)}, substituted alkyl_{(C≤8)}, or a group of the formula:

—Y₂—C(O)Rₑ
wherein:
   Y₂ is alkanediyl_{(C≤12)} or substituted alkanediyl_{(C≤12)}; and
   Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.
   In some embodiments, the compounds are further defined as:

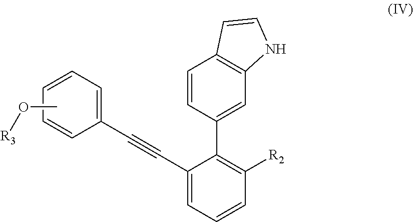

wherein:
   R₂ is —C(O)Rₐ, wherein:
      Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
   —NR_{b}C(X₂)NR_{c}R_{c}′, wherein
      R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
   X₂ is O or NR_{d}, wherein R_{d} is hydrogen, alkyl_{(C≤8)}, or substituted alkyl_{(C≤8)};
   R₃ is hydrogen, alkyl_{(C≤8)}, substituted alkyl_{(C≤8)}, or a group of the formula:

—Y₂—C(O)Rₑ
wherein:
   Y₂ is alkanediyl_{(C≤12)} or substituted alkanediyl_{(C≤12)}; and
   Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

In some embodiments, the compounds are further defined as:

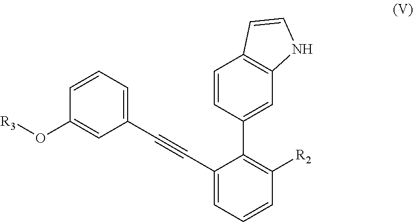

wherein:
   R₂ is —C(O)Rₐ, wherein:
      Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
   —NR_{b}C(X₂)NR_{c}R_{c}′, wherein
      R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
   X₂ is O or NR_{d}, wherein R_{d} is hydrogen, alkyl_{(C≤8)}, or substituted alkyl_{(C≤8)};
   R₃ is hydrogen, alkyl_{(C≤8)}, substituted alkyl_{(C≤8)}, or a group of the formula:

—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

In some embodiments, R₁ is hydrogen. In some embodiments, Y₁ is alkynediyl_{(C≤12)} or substituted alkynediyl_{(C≤12)}. In some embodiments, Y₁ is alkynediyl_{(C≤12)} such as ethynediyl. In some embodiments, X₁ is arenediyl_{(C≤12)} or substituted arenediyl_{(C≤12)}. In some embodiments, X₁ is arenediyl_{(C≤12)} such as benzenediyl. In some embodiments, X₁ is 1,3-benzenediyl.

In some embodiments, R₂ is —C(O)Rₐ. In some embodiments, Rₐ is hydroxy. In some embodiments, Rₐ is alkoxy_{(C≤8)} or substituted alkoxy_{(C≤8)}. In some embodiments, Rₐ is alkoxy_{(C≤8)} such as methoxy or t-butyloxy. In some embodiments, R₂ is —NR_{b}C(X₂)NR_{c}R_{c}′. In some embodiments, R_{b} is hydrogen. In some embodiments, X₂ is O. In some embodiments, R_{c} is hydrogen. In some embodiments, R_{c}′ is an alkyl_{(C≤12)} substituted with one or more mono- or divalent protected amines or a substituted alkyl_{(C≤12)} substituted with one or more mono- or divalent protected amines. In some embodiments, R_{c}′ is an alkyl_{(C≤12)} substituted with one or more mono- or divalent protected amines. In some embodiments, R_{c}′ is an alkyl_{(C≤12)} substituted with one monovalent protected amine. In some embodiments, R_{c}′ is substituted with a Boc protected amine group such as N-Boc-aminopentyl.

In some embodiments, R₃ is hydrogen. In some embodiments, R₃ is —Y₂—C(O)Rₑ. In some embodiments, Y₂ is alkanediyl_{(C≤12)}. In some embodiments, Y₂ is methylene. In some embodiments, Rₑ is alkoxy_{(C≤8)} or substituted alkoxy_{(C≤8)}. In some embodiments, Rₑ is alkoxy_{(C≤8)}. In some embodiments, Rₑ is t-butyloxy.

In some embodiments, the compounds are further defined as:

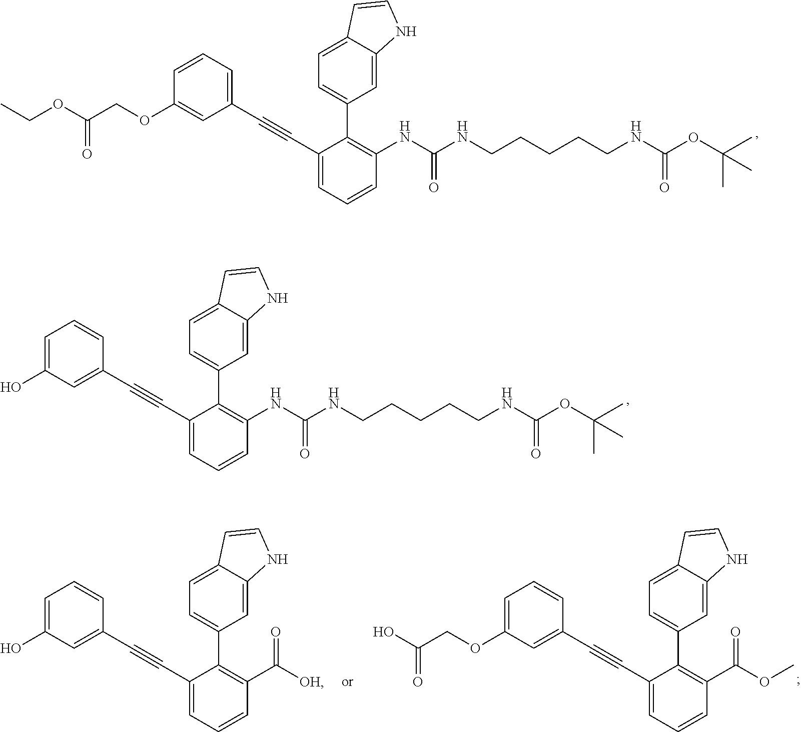 [SMILES: CCOC(=O)COc1cccc(C#Cc2cccc(NC(=O)NCCCCCNC(=O)OC(C)(C)C)c2-c2ccc3cc[nH]c3c2)c1 ; CC(C)(C)OC(=O)NCCCCCNC(=O)Nc1cccc(C#Cc2cccc(O)c2)c1-c1ccc2cc[nH]c2c1 ; O=C(O)c1cccc(C#Cc2cccc(O)c2)c1-c1ccc2cc[nH]c2c1 ; COC(=O)c1cccc(C#Cc2cccc(OCC(=O)O)c2)c1-c1ccc2cc[nH]c2c1]

or a pharmaceutically acceptable salt thereof.

In another aspect, the present disclosure provides pharmaceutical compositions comprising:
(A) a compound described herein; and
(B) a E3 ligase ligand;
   wherein the compound and the E3 ligase ligand are covalently linked.

In some embodiments, the E3 ligase ligand is further defined as: is a Cereblon (CRBN) ligand, a Von-Hippel Lindau (VHL) ligand, a mouse double minute 2 homolog (MDM2) ligand, or a cellular inhibitor of apoptosis protein 1 (cIAP1) ligand. In some embodiments, E3 ligase ligand is a CRBN ligand.

In some embodiments, E3 ligase ligand is a group of the formula:

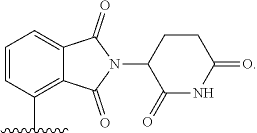 [SMILES: *c1cccc2c1C(=O)N(C1CCC(=O)NC1=O)C2=O]

In some embodiments, E3 ligase ligand is a VHL ligand. In some embodiments, E3 ligase ligand is a group of the formula:

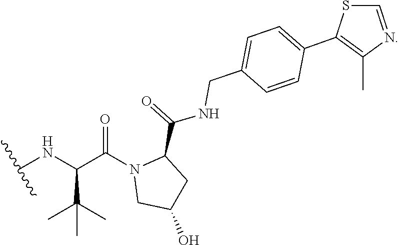 [SMILES: *N[C@@H](C(=O)N1C[C@@H](O)C[C@@H]1C(=O)NCc1ccc(-c2scnc2C)cc1)C(C)(C)C]

In some embodiments, E3 ligase ligand is a MDM2 ligand. In some embodiments, E3 ligase ligand is a group of the formula:

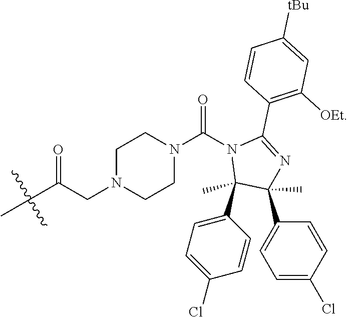 [SMILES: *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1]

In another aspect, the present disclosure provides pharmaceutical compositions comprising:
(A) a compound or composition described herein; and
(B) a pharmaceutically acceptable excipient.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical composition is formulated for oral, intraarterial, or intravenous administration. In some embodiments, the pharmaceutical composition is formulated as a unit dose.

In yet another aspect, the present disclosure provides methods of treating or preventing a disease or disorder in a patient in need thereof comprising administering to the patient a therapeutically effective amount of a compound or composition described herein.

In some embodiments, the disease or disorder is associated with an Epstein-Barr virus infection. In some embodiments, the disease or disorder is associated with the presence of Epstein-Barr virus nuclear antigen 1 (EBNA1).

In some embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is an epithelial cancer such as a nasopharyngeal carcinoma, lymphoepithelioma-like carcinoma, or a gastric cancer. In other embodiments, the cancer is a lymphoid cancer such as Burkitt lymphoma, Hodgkin lymphoma, HIV-associated non-Hodgkin lymphoma, Diffuse large B-cell lymphoma, lymphomatoid granulomatosis, NK/T-cell lymphoma, natural killer cell leukemia, natural killer cell lymphoma, angioimmunoblastic T-cell lymphoma, enteropathy-type T-cell lymphoma, cutaneous T-cell lymphoproliferative disorder, γδ T-cell lymphoma, peripheral T-cell lymphoma, or T-cell lymphoproliferative disorders. In other embodiments, the cancer is breast cancer, thyroid cancer, salivary gland cancer, or hepatobiliary cancer.

In other embodiments, the disease or disorder is an autoimmune disease or disorder. In some embodiments, the autoimmune disease or disorder is a systemic autoimmune disease such as rheumatoid arthritis, Sjögren's syndrome, systemic lupus erythematosus, systemic scleroderma, polymyositis, systemic sclerosis, or mixed connective tissue disease. In other embodiments, the autoimmune disease is multiple sclerosis.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Strategy using structure guided approaches to incorporate linkers for the synthesis of bi-functional molecules.

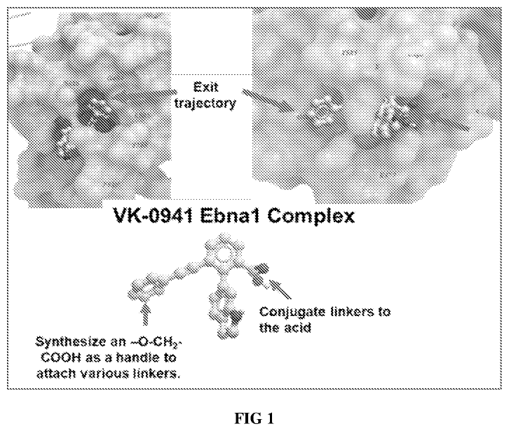

FIG. 2—Synthesis of the orthogonally protected di-ester FSM-4-213 as a key intermediate for construction of bi-functional molecules.

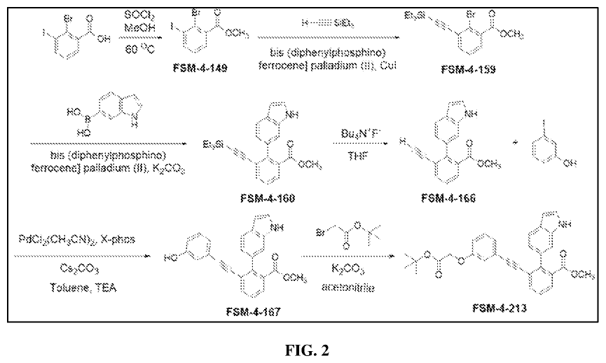

FIG. 3—Synthesis of an Mdm2 recruiting ligand suitable for conjugation.

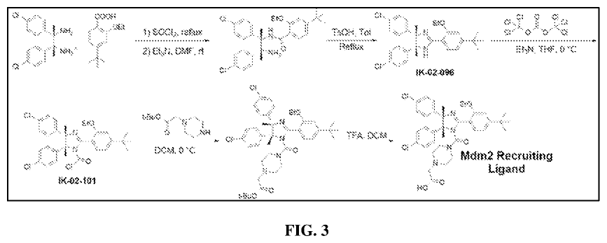

FIG. 4—Reference EBNA-1 ligands and modified EBNA-1 ligands which incorporate a ligand for coupling to an E3 ligase recruiting molecule.

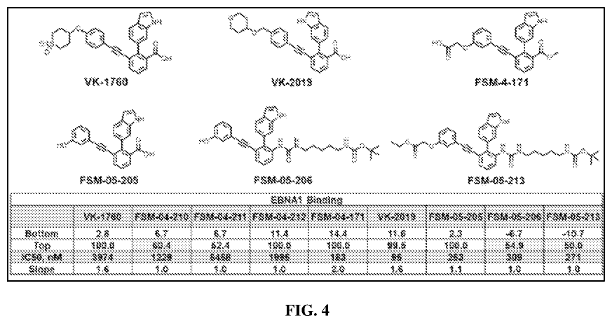

FIG. 5—A. Signal linearity for the SNU-719 cell line is determined for various cell number and antibody concentration. B. Plate image for the primary antibody. C. Calculation for assay signal to noise where the parameters in red are used for the assay. D. Signal linearity for the C6661 cell line is determined for various cell number and antibody concentration. E. Plate image for the primary antibody. F. Calculation for assay signal to noise where the parameters in red are used for the assay.

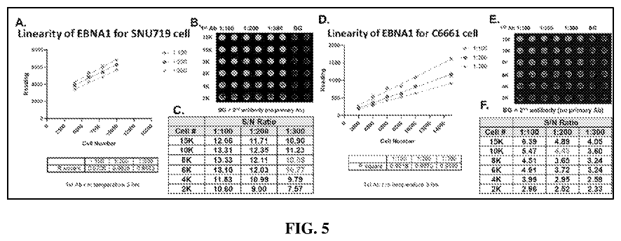

### DESCRIPTION OF ILLUSTRATIVE EMBODIMENTS

Provided herein are compounds and compositions that may be used treat one or more conditions associated with an infection of an Epstein-Barr virus. In particular, the compounds may bind to EBNA1 and cause degradation of this protein. These compounds may be used to treat cancers associated with or caused by an Epstein-Barr virus or an autoimmune disease. The compounds described herein may be linked to a E3 ligase ligand to form a PROTAC that degrades EBNA1. These and more details will be described below.

### I. Compounds and Formulations Thereof

### A. Compounds of the Present Disclosure

The compounds of the present disclosure are shown, for example, in the summary section above and in Table 1 and the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1

| Compounds of the Present Disclosure | |
|---|---|
| Compound | |
| ID | Structure |
| FSM-05-213 | 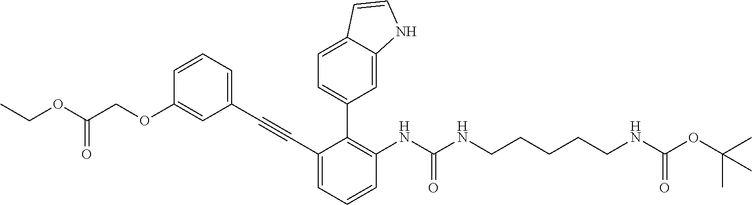 [SMILES: CCOC(=O)COc1cccc(C#Cc2cccc(NC(=O)NCCCCCNC(=O)OC(C)(C)C)c2-c2ccc3cc[nH]c3c2)c1] |
| FSM-05-206 | 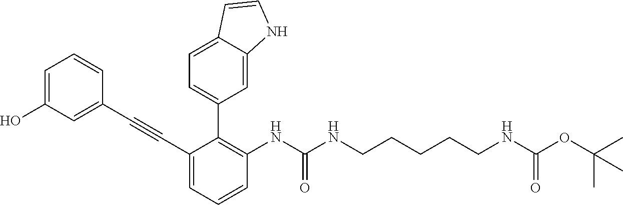 [SMILES: CC(C)(C)OC(=O)NCCCCCNC(=O)Nc1cccc(C#Cc2cccc(O)c2)c1-c1ccc2cc[nH]c2c1] |
| FSM-05-205 | 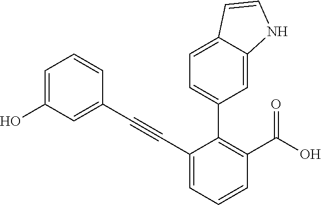 [SMILES: O=C(O)c1cccc(C#Cc2cccc(O)c2)c1-c1ccc2cc[nH]c2c1] |
| FSM-4-171 | 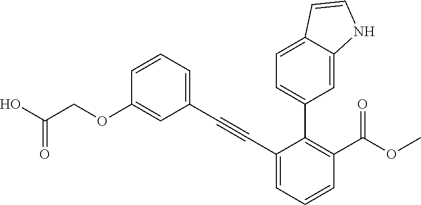 [SMILES: COC(=O)c1cccc(C#Cc2cccc(OCC(=O)O)c2)c1-c1ccc2cc[nH]c2c1] |

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### B. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22 (3): 659-661, 2008, which is incorporated herein by reference):

HED (mg/kg)=Animal dosee (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount is from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### II. Methods of Treatment and Combination Therapies

### A. Methods of Treatment

In particular the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, the amount of the compounds used is calculated to be from about 0.01 mg to about 10,000 mg/day. In some embodiments, the amount is from about 1 mg to about 1,000 mg/day. In some embodiments, the compounds may be administered for 1 day to 20 days. In further embodiments, it is contemplated that the compounds may be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days, or any range derivable therein. In some embodiments, the compounds may be administered for between 3 and 5 days, inclusive. In some embodiments, the compounds may be administered once. It is also contemplated that in some embodiments, the compounds disclosed herein may be administered two or more times. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### B. Combination Therapies

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

To treat diseases or disorders using the methods and compositions of the present disclosure, one would generally contact a cell or a subject with a compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent. In some embodiments, the compounds of the present disclosure or any therapies used in conjunction with the compounds of the present disclosure may be administered in a less than therapeutically effective dose when used either alone or in combination.

Non-limiting examples of such combination therapy include combination of one or more compounds of the invention with another anti-inflammatory agent, an immunosuppressant agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

It is also conceivable that more than one administration of either the compound or the other therapy will be desired. Various combinations may be employed, where a compound of the present disclosure is “A,” and the other therapy is “B,” as exemplified below:

| | | | | | | |
|---|---|---|---|---|---|---|
| A/B/A | B/A/B B/B/A | A/A/B | B/A/A | A/B/B | B/B/B/A A | B/B/A/B |
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are also contemplated.

### III. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C—O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

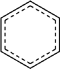

covers, for example,

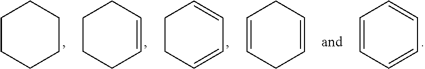

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

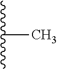

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

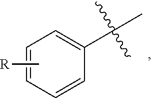

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

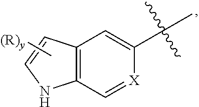

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n +2 electrons in a fully conjugated cyclic π system. An aromatic compound or chemical group may be depicted as a single resonance structure; however, depiction of one resonance structure is taken to also refer to any other resonance structure. For example:

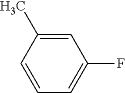

is also taken to refer to

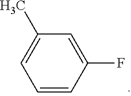

Aromatic compounds may also be depicted using a circle to represent the delocalized nature of the electrons in the fully conjugated cyclic π system, two non-limiting examples of which are shown below:

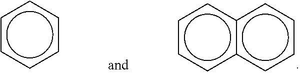

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂-(methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

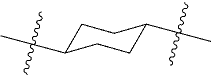 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” refers to a divalent unsaturated aliphatic group, with two carbon atoms as points of attachment, a linear or branched acyclic structure, at least one nonaromatic carbon-carbon triple bond, no carbon-carbon double bonds, and no atoms other than carbon and hydrogen. The groups —C≡C—, —C≡CCH₂—, and —CH₂C═CCH₂— are non-limiting examples of alkynediyl groups. It is noted that while the alkynediyl group is aliphatic, once connected at both ends, this group is not precluded from forming part of an aromatic structure. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

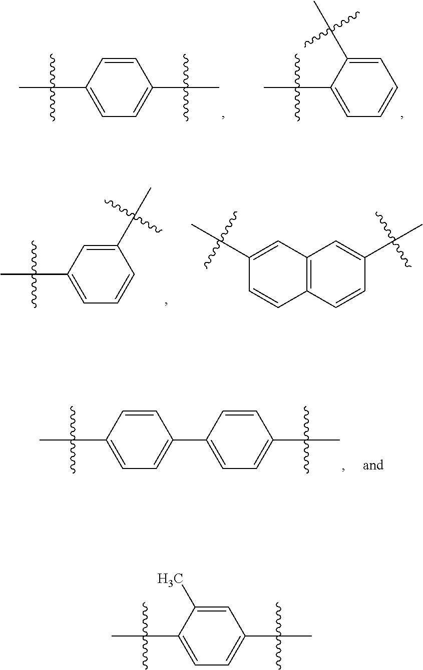

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl (cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃.

An “amine protecting group” or “amino protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxy-benzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monovalently protected amino group” and PG_{DA} is a divalent amine protecting group as described above, which may also be described as a “divalently protected amino group”.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

As used herein, the term “functional group” refers to any chemical group or substituent covalently bound to a core structure. Functional groups may include but are not limited to hydrogen, alkyl, alkenyl, alkynyl, cycloalkyl, heterocycloalkyl, aryl, heteroaryl, aralkyl, —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, —S(O)₂NH₂ or a combination or substituted version of any of these groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API)(also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly (lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API)(defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “ubiquitin ligase ligand” or “E3 ligase ligand” refers to a chemical group capable of binding ubiquitin ligase. Ubiquitin ligase (also called an E3 ubiquitin ligase) is a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. E3 ligases interact with both the target protein and the E2 enzyme, and so impart substrate specificity to the E2. Commonly, E3s polyubiquitinate their substrate with Lys48-linked chains of ubiquitin, targeting the substrate for destruction by the proteasome. However, one of skill in the art recognizes that many other types of linkages are possible and that each may alter a protein's activity, interactions, or localization. Ubiquitination by E3 ligases regulates diverse areas such as cell trafficking, DNA repair, and signaling and is of profound importance in cell biology. E3 ligases are also key players in cell cycle control, mediating the degradation of cyclins, as well as cyclin dependent kinase inhibitor proteins. The human genome encodes over 600 putative E3 ligases, allowing for tremendous diversity in substrates. Non-limiting examples of ubiquitin ligase ligands include the von Hippel-Lindau (VHL) ligand, a cIAP1 ligand, a MDM2 ligand, a CRBN ligand, a CUL2 ligand, or other ligand which binds to one or more of the proteins of the ubiquitin protein complex especially the E3 component of this complex.

The term “EBNA-1 targeting ligand” refers to a chemical group capable of inhibiting Epstein-Barr Nuclear Antigen-1 (EBNA-1) at maximum IC₅₀ value of 6000 nM. EBNA-1 is essential for the persistence of the EBV episomal genomes due to its important roles in both the replication and the mitotic segregation of the EBV episomes (Sivachandran et al., 2008). EBNA-1 is also the only viral protein required for the replication of the oriP-based expression vector and does not elicit a cellular immune response, as it has developed an efficient mechanism to bypass the processing required for presentation of its antigens on MHC class I molecules (Levitskaya et al., 1997). Further, EBNA-1 can act in trans to enhance expression of the cloned gene, inducing expression of a cloned gene up to 100-fold in some cell lines (Langle-Rouault et al., 1998).

Ubiquitin ligase (also called an E3 ubiquitin ligase or E3 ligase) is a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. E3 ligases interact with both the target protein and the E2 enzyme, and so impart substrate specificity to the E2. Commonly, E3s polyubiquitinate their substrate with Lys48-linked chains of ubiquitin, targeting the substrate for destruction by the proteasome. However, one of skill in the art recognizes that many other types of linkages are possible and that each may alter a protein's activity, interactions, or localization. Ubiquitination by E3 ligases regulates diverse areas such as cell trafficking, DNA repair, and signaling and is of profound importance in cell biology. E3 ligases are also key players in cell cycle control, mediating the degradation of cyclins, as well as cyclin dependent kinase inhibitor proteins. The human genome encodes over 600 putative E3 ligases, allowing for tremendous diversity in substrates. Non-limiting examples of ubiquitin ligase ligands include the von Hippel-Lindau (VHL) ligand, a cIAP1 ligand, a MDM2 ligand, a CRBN ligand, a CUL2 ligand, or other ligand which binds to one or more of the proteins of the ubiquitin protein complex especially the E3 component of this complex.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### I. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synthetic Methodology

### A. Synthesis of the Orthogonally Protected Di-Ester FSM-4-213

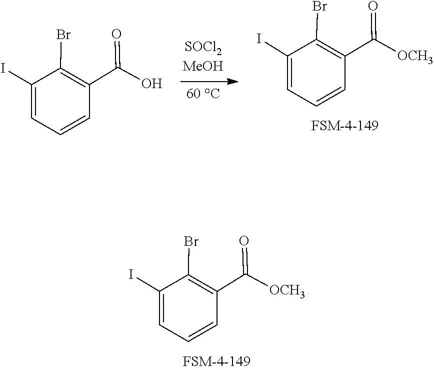 [SMILES: O=C(O)c1cccc(I)c1Br ; COC(=O)c1cccc(I)c1Br ; COC(=O)c1cccc(I)c1Br]

Methyl 2-bromo-3-iodobenzoate:

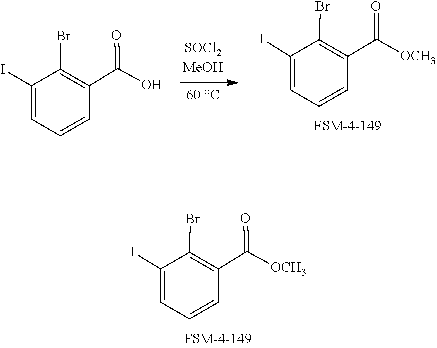 [SMILES: O=C(O)c1cccc(I)c1Br ; COC(=O)c1cccc(I)c1Br ; COC(=O)c1cccc(I)c1Br]

Methyl 2-bromo-3-iodobenzoate:

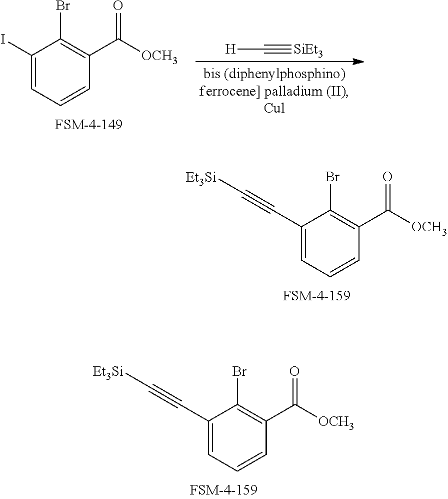

Methyl 2-bromo-3-((triethylsilyl) ethynyl)benzoate

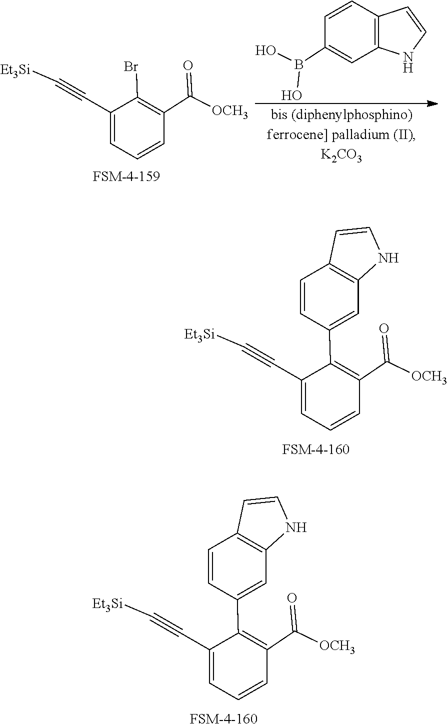

Methyl 2-(1H-indol-6-yl)-3-((triethylsilyl) ethynyl)benzoate

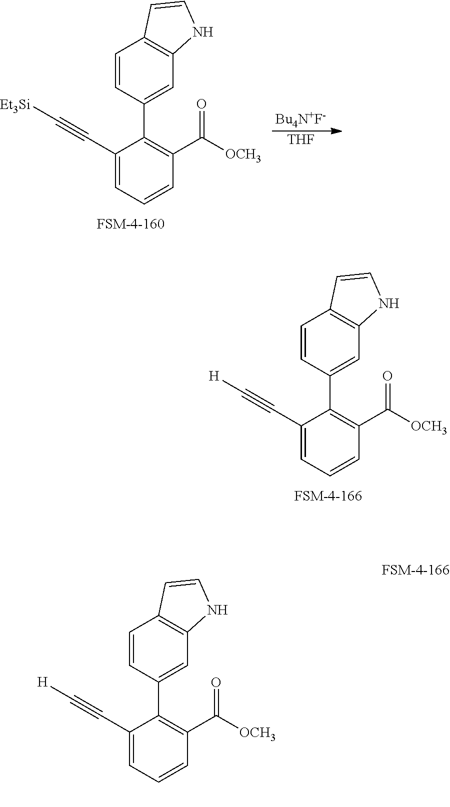

Methyl 3-ethynyl-2-(1H-indol-6-yl)benzoate

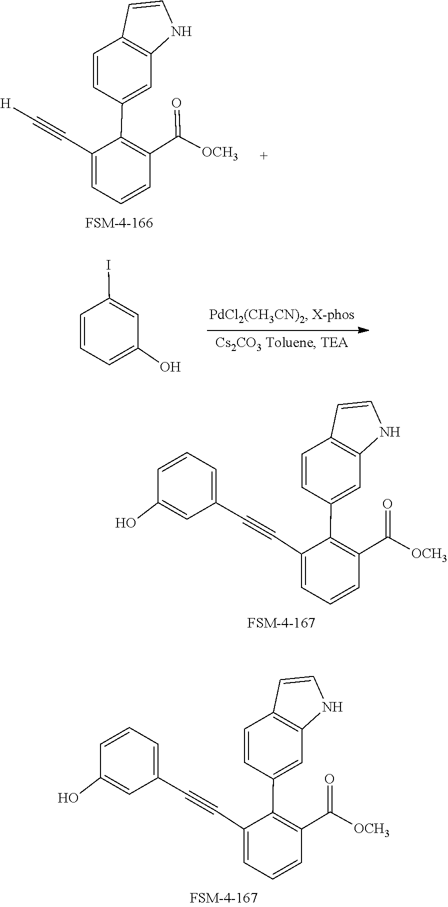 [SMILES: C#Cc1cccc(C(=O)OC)c1-c1ccc2cc[nH]c2c1 ; Oc1cccc(I)c1 ; COC(=O)c1cccc(C#Cc2cccc(O)c2)c1-c1ccc2cc[nH]c2c1 ; COC(=O)c1cccc(C#Cc2cccc(O)c2)c1-c1ccc2cc[nH]c2c1]

Methyl 3-((3-hydroxyphenyl) ethynyl)-2-(1H-indol-6-yl)benzoate

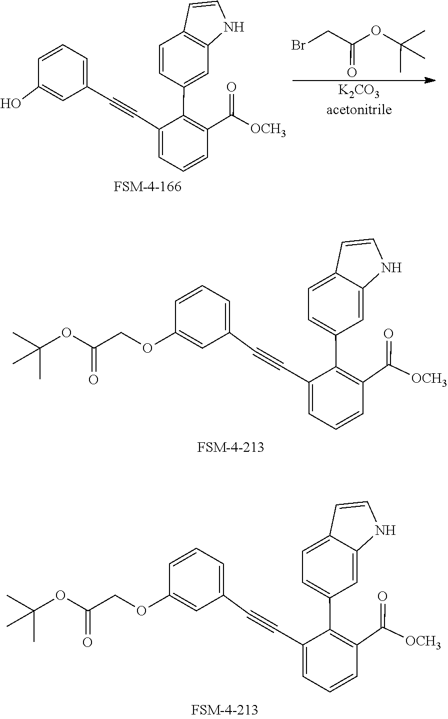

Methyl 3-((3-(2-(tert-butoxy)-2-oxoethoxy)phenyl) ethynyl)-2-(1H-indol-6-yl)benzoate

### REFERENCES

The following references, to the extent that they provide exemplary procedural or other details supplementary to those set forth herein, are specifically incorporated herein by reference:
Andrei et al., Mol., 24 (5): 997, 2019.
Frappier L. et al., Scient., 438204, 2012.
Boudreault et al., Vir Jour., 16:29, 2019.
Gruhne et al., Proc Natl Acad Sci USA., 106:2313-2318, 2009.
Schultz et al., Proc Natl Acad Sci USA., 106 (7): 2091-2092, 2009.
Messick et al., Sci Trans Med., 11:482, 2019.
Jakhmola et al., ACS Chem Neurosci., 12 (16): 3060-3072.
Handbook of Pharmaceutical Salts: Properties, and Use, Stahl and Wermuth Eds., Verlag Helvetica Chimica Acta, 2002.
Lu et al., J. Clin. Invest., 121 (10): 4015-29, 2011.
Reagan-Shaw et al., FASEB J., 22 (3): 659-661, 2008.
Sivachandran et al., PLoS Pathog., 4:10, 2008.
Levitskaya et al., Proc Natl Acad Sci USA., 94 (23): 12616-12621, 1997.
Langle-Rouault et al., J Virol., 72 (7): 6181-6185, 1998.

What is claimed:

1. A compound of the formula: 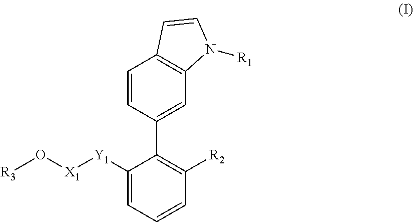
wherein:
R₁ is hydrogen, alkyl_{(C≤8)}, or substituted alkyl_{(C≤8)};
R₂ is —C(O)Rₐ, wherein:
Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
X₂ is O or NR_{d}, wherein R_{d} is hydrogen, alkyl_{(C≤8)}, or substituted alkyl_{(C≤8)};
Y₁ is alkanediyl_{(C≤12)}, cycloalkanediyl_{(C≤12)}, alkenediyl_{(C≤12)}, alkynediyl_{(C≤12)}, or a substituted version thereof;
X₁ is arenediyl_{(C≤12)}, heteroarenediyl_{(C≤12)}, or a substituted version thereof; and
R₃ is hydrogen, alkyl_{(C≤8)}, substituted alkyl_{(C≤8)}, or a group of the formula:
—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

2. The compound of claim 1, further defined as: 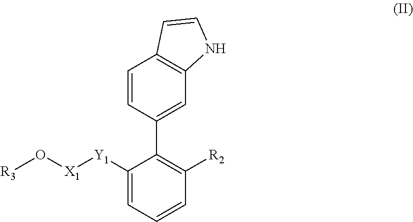
wherein:
R₂ is —C(O)Rₐ, wherein:
Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
X₂ is O or NR_{d}, wherein R_{d} is hydrogen, alkyl_{(C≤8)}, or substituted alkyl_{(C≤8)};
Y₁ is alkanediyl_{(C≤12)}, cycloalkanediyl_{(C≤12)}, alkenediyl_{(C≤12)}, alkynediyl_{(C≤12)}, or a substituted version thereof;
X₁ is arenediyl_{(C≤12)}, heteroarenediyl_{(C≤12)}, or a substituted version thereof; and
R₃ is hydrogen, alkyl_{(C≤8)}, substituted alkyl_{(C≤8)}, or a group of the formula:
—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

3. The compound of either claim 1 or claim 2, further defined as: 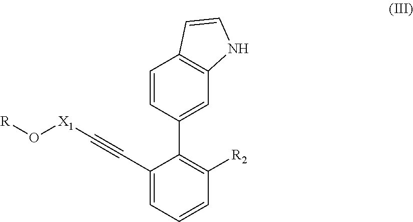
wherein:
R₂ is —C(O)Rₐ, wherein:
Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
X₂ is O or NR_{d}, wherein R_{d} is hydrogen, alkyl_{(C≤8)}, or substituted alkyl_{(C≤8)};
X₁ is arenediyl_{(C≤12)}, heteroarenediyl_{(C≤12)}, or a substituted version thereof; and
R₃ is hydrogen, alkyl_{(C≤8)}, substituted alkyl_{(C≤8)}, or a group of the formula:
—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

4. The compound according to any one of claims 1-3, further defined as: 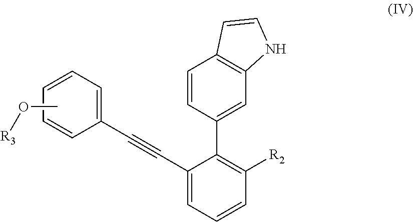
wherein:
R₂ is —C(O)Rₐ, wherein:
Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
X₂ is O or NR_{d}, wherein R_{d} is hydrogen, alkyl_{(C≤8)}, or substituted alkyl_{(C≤8)};
R₃ is hydrogen, alkyl_{(C≤8)}, substituted alkyl_{(C≤8)}, or a group of the formula:
—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

5. The compound according to any one of claims 1-4, further defined as: 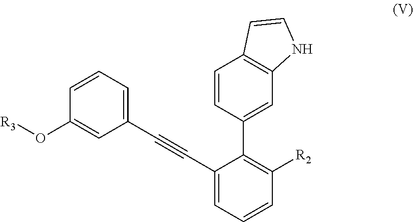
wherein:
R₂ is —C(O)Rₐ, wherein:
Rₐ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group; or
—NR_{b}C(X₂)NR_{c}R_{c}′, wherein
R_{b}, R_{c}, and R_{c}′ are each independently hydrogen, alkyl_{(C≤12)}, substituted alkyl_{(C≤12)}, an alkyl_{(C≤12)} substituted with one or more mono- or divalent protected amines, a monovalent amine protecting group, or R_{c} and R_{c}′ are a single divalent amine protecting group, and
X₂ is O or NR_{d}, wherein R_{d} is hydrogen, alkyl_{(C≤8)}, or substituted alkyl_{(C≤8)};
R₃ is hydrogen, alkyl_{(C≤8)}, substituted alkyl_{(C≤8)}, or a group of the formula:
—Y₂—C(O)Rₑ
wherein:
Y₂ is alkanediyl_{(C≤12)} or substituted alkanediyl_{(C≤12)}; and
Rₑ is amino, hydroxy, alkoxy_{(C≤8)}, substituted alkoxy_{(C≤8)}, alkylamino_{(C≤8)}, substituted alkylamino_{(C≤8)}, dialkylamino_{(C≤8)}, substituted dialkylamino_{(C≤8)}, or a carboxylic acid protecting group;
or a pharmaceutically acceptable salt thereof.

6. The compound of claim 1, wherein R₁ is hydrogen.

7. The compound of either claim 1, 2, or 6, wherein Y₁ is alkynediyl_{(C≤12)} or substituted alkynediyl_{(C≤12)}.

8. The compound of claim 7, wherein Y₁ is alkynediyl_{(C≤12)}.

9. The compound of either claim 7 or claim 8, wherein Y₁ is ethynediyl.

10. The compound according to any one of claims 1-3 and 6-9, wherein X₁ is arenediyl_{(C≤12)} or substituted arenediyl_{(C≤12)}.

11. The compound of claim 10, wherein X₁ is arenediyl_{(C≤12)}.

12. The compound of either claim 10 or claim 11, wherein X₁ is benzenediyl.

13. The compound according to any one of claims 10-12, wherein X₁ is 1,3-benzenediyl.

14. The compound according to any one of claims 1-13, wherein R₂ is —C(O)Rₐ.

15. The compound according to any one of claims 1-14, wherein Rₐ is hydroxy.

16. The compound according to any one of claims 1-14, wherein Rₐ is alkoxy_{(C≤8)} or substituted alkoxy_{(C≤8)}.

17. The compound of claim 16, wherein Rₐ is alkoxy_{(C≤8)}.

18. The compound of either claim 16 or claim 17, wherein Rₐ is methoxy or t-butyloxy.

19. The compound according to any one of claims 1-13, wherein R₂ is —NR_{b}C(X₂)NR_{c}R_{c}′.

20. The compound according to any one of claims 1-13 and 19, wherein R_{b} is hydrogen.

21. The compound according to any one of claims 1-13, 19, and 20, wherein X₂ is O.

22. The compound according to any one of claims 1-13 and 19-21, wherein R_{c} is hydrogen.

23. The compound according to any one of claims 1-13 and 19-22, wherein R_{c}′ is an alkyl_{(C≤12)} substituted with one or more mono- or divalent protected amines or a substituted alkyl_{(C≤12)} substituted with one or more mono- or divalent protected amines.

24. The compound of claim 23, wherein R_{c}′ is an alkyl_{(C≤12)} substituted with one or more mono- or divalent protected amines.

25. The compound of either claim 23 or claim 24, wherein R_{c}′ is an alkyl_{(C≤12)} substituted with one monovalent protected amine.

26. The compound according to any one of claims 23-25, wherein R_{c}′ is substituted with a Boc protected amine group.

27. The compound according to any one of claims 23-26, wherein R_{c}′ is N-Boc-aminopentyl.

28. The compound according to any one of claims 1-27, wherein R₃ is hydrogen.

29. The compound according to any one of claims 1-27, wherein R₃ is —Y₂—C(O)Rₑ.

30. The compound of claim 29, wherein Y₂ is alkanediyl_{(C≤12)}.

31. The compound of claim 30, wherein Y₂ is methylene.

32. The compound according to any one of claims 29-31, wherein Rₑ is alkoxy_{(C≤8)} or substituted alkoxy_{(C≤8)}.

33. The compound according to any one of claims 29-32, wherein Rₑ is alkoxy_{(C≤8)}.

34. The compound according to any one of claims 29-33, wherein Rₑ is t-butyloxy.

35. The compound according to any one of claims 1-34, wherein the compound is further defined as: 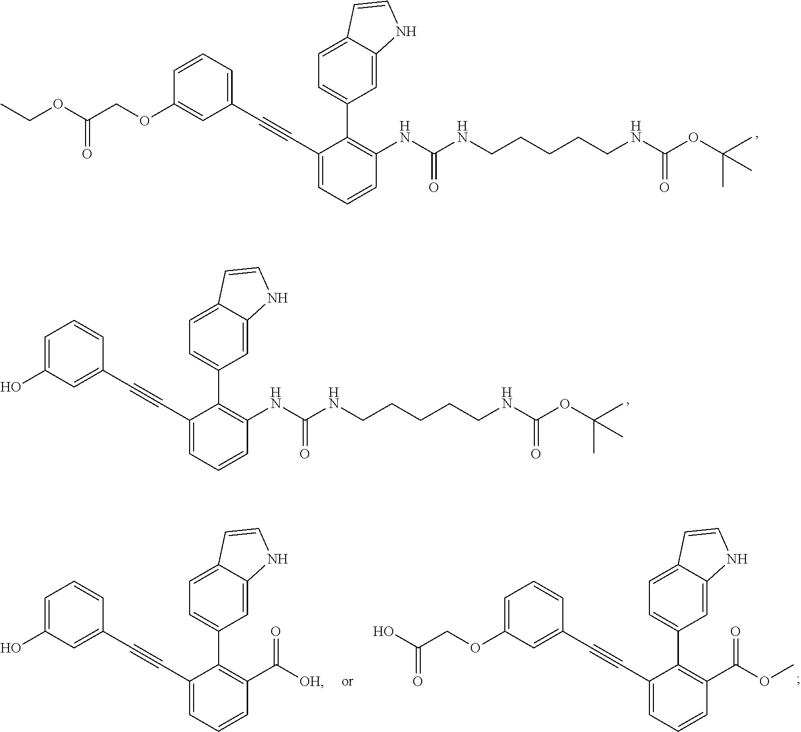 [SMILES: CCOC(=O)COc1cccc(C#Cc2cccc(NC(=O)NCCCCCNC(=O)OC(C)(C)C)c2-c2ccc3cc[nH]c3c2)c1 ; CC(C)(C)OC(=O)NCCCCCNC(=O)Nc1cccc(C#Cc2cccc(O)c2)c1-c1ccc2cc[nH]c2c1 ; O=C(O)c1cccc(C#Cc2cccc(O)c2)c1-c1ccc2cc[nH]c2c1 ; COC(=O)c1cccc(C#Cc2cccc(OCC(=O)O)c2)c1-c1ccc2cc[nH]c2c1]
or a pharmaceutically acceptable salt thereof.

36. A composition comprising:
(A) a compound according to any one of claims 1-35; and
(B) a E3 ligase ligand;
wherein the compound and the E3 ligase ligand are covalently linked.

37. The composition of claim 36, wherein the E3 ligase ligand is further defined as: is a Cereblon (CRBN) ligand, a Von-Hippel Lindau (VHL) ligand, a mouse double minute 2 homolog (MDM2) ligand, or a cellular inhibitor of apoptosis protein 1 (cIAP1) ligand.

38. The composition of claim 37, wherein E3 ligase ligand is a CRBN ligand.

39. The composition of claim 38, wherein E3 ligase ligand is a group of the formula:

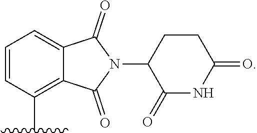 [SMILES: *c1cccc2c1C(=O)N(C1CCC(=O)NC1=O)C2=O]

40. The composition of claim 37, wherein E3 ligase ligand is a VHL ligand.

41. The composition of claim 40, wherein E3 ligase ligand is a group of the formula:

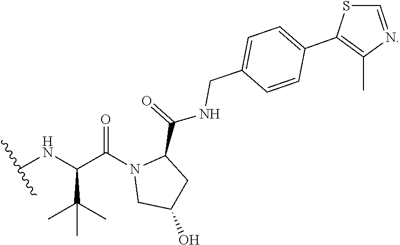 [SMILES: *N[C@@H](C(=O)N1C[C@@H](O)C[C@@H]1C(=O)NCc1ccc(-c2scnc2C)cc1)C(C)(C)C]

42. The composition of claim 37, wherein E3 ligase ligand is a MDM2 ligand.

43. The composition of claim 42, wherein E3 ligase ligand is a group of the formula:

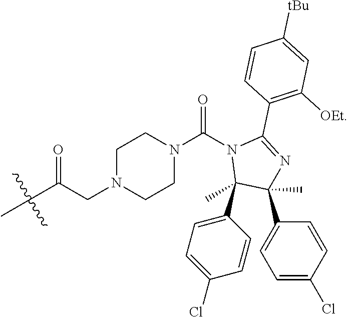 [SMILES: *C(=O)CN1CCN(C(=O)N2C(c3ccc(C(C)(C)C)cc3OCC)=N[C@@](C)(c3ccc(Cl)cc3)[C@@]2(C)c2ccc(Cl)cc2)CC1]

44. A pharmaceutical composition comprising:
(A) a compound or composition according to any one of claims 1-43; and
(B) a pharmaceutically acceptable excipient.

45. The pharmaceutical composition of claim 44, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

46. The pharmaceutical composition of claim 45, wherein the pharmaceutical composition is formulated for oral, intraarterial, or intravenous administration.

47. The pharmaceutical composition according to any one of claims 44-46, wherein the pharmaceutical composition is formulated as a unit dose.

48. A method of treating or preventing a disease or disorder in a patient in need thereof comprising administering to the patient a therapeutically effective amount of a compound or composition according to any one of claims 1-47.

49. The method of claim 48, wherein the disease or disorder is associated with an Epstein-Barr virus infection.

50. The method of either claim 48 or claim 49, wherein the disease or disorder is associated with the presence of Epstein-Barr virus nuclear antigen 1 (EBNA1).

51. The method according to any one of claims 48-50, wherein the disease or disorder is cancer.

52. The method of claim 51, wherein the cancer is a carcinoma, sarcoma, lymphoma, leukemia, melanoma, mesothelioma, multiple myeloma, or seminoma.

53. The method of claim 51, wherein the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

54. The method according to any one of claims 51-53, wherein the cancer is an epithelial cancer.

55. The method of claim 54, wherein the epithelial cancer is a nasopharyngeal carcinoma, lymphoepithelioma-like carcinoma, or a gastric cancer.

56. The method according to any one of claims 51-53, wherein the cancer is a lymphoid cancer.

57. The method of claim 56, wherein the lymphoid cancer is Burkitt lymphoma, Hodgkin lymphoma, HIV-associated non-Hodgkin lymphoma, Diffuse large B-cell lymphoma, lymphomatoid granulomatosis, NK/T-cell lymphoma, natural killer cell leukemia, natural killer cell lymphoma, angioimmunoblastic T-cell lymphoma, enteropathy-type T-cell lymphoma, cutaneous T-cell lymphoproliferative disorder, γδ T-cell lymphoma, peripheral T-cell lymphoma, or T-cell lymphoproliferative disorders.

58. The method according to any one of claims 51-53, wherein the cancer is breast cancer, thyroid cancer, salivary gland cancer, or hepatobiliary cancer.

59. The method according to any one of claims 48-50, wherein the disease or disorder is an autoimmune disease or disorder.

60. The method of claim 59, wherein the autoimmune disease or disorder is a systemic autoimmune disease.

61. The method of either claim 59 or claim 60, wherein the autoimmune disease or disorder is rheumatoid arthritis, Sjögren's syndrome, systemic lupus erythematosus, systemic scleroderma, polymyositis, systemic sclerosis, or mixed connective tissue disease.

62. The method of claim 59, wherein the autoimmune disease is multiple sclerosis.
